(19) United States
(12) Patent Application Publication
(10) Pub. No.: US 2022/0395225 A1
(43) Pub. Date: Dec. 15, 2022

(54) MODULAR WRISTBAND AND SENSOR SYSTEM

(71) Applicant: Rajant Health Inc., Malvern, PA (US)

(72) Inventors: Robert J. Schena, Malvern, PA (US); Emma K. Murray, Malvern, PA (US); Giana J. Schena, Malvern, PA (US); Spyridon Manganas, Malvern, PA (US); Muthukumaran Chandrasekaran, Malvern, PA (US); Muhammad Anjum, Malvern, PA (US)

(21) Appl. No.: 17/806,475

(22) Filed: Jun. 10, 2022

Related U.S. Application Data
(60) Provisional application No. 63/209,298, filed on Jun. 10, 2021.

(51) Int. Cl. A61B 5/00; A61B 5/0533; A61B 5/0535; A61B 5/0205; A61B 5/024; A61B 5/318; G16H 40/67

(52) U.S. Cl. CPC A61B 5/681; A61B 5/0533; A61B 5/0535; A61B 5/0205; A61B 5/02416; A61B 5/318; G16H 40/67; A61B 2562/0219

(57) ABSTRACT

A modular wristband and sensor system and method of using the same are disclosed. The system uses top and bottom sensor modules that contain conductive ports for connection to wristbands' conductive ports. The wristbands' conductive ports are electrically connected to wires embedded within each wristband segment. These allow for the transfer of data and power between the bands and the top and bottom sensor modules. Having power and data conducted through wristbands into sensors makes it possible for wristband sensors to have swappable bands while maintaining connectivity. Other embodiments include a wristband that includes swappable top and bottom sensor modules communicating wirelessly with each other.

### CROSS REFERENCE TO RELATED APPLICATION(S)

This application claims priority to U.S. Provisional Patent Application Serial No. 63/209,298, filed on Jun. 10, 2021, which is hereby incorporated herein by reference in its entirety.

### GOVERNMENT SPONSORSHIP

None

### FIELD OF THE INVENTION

Embodiments are in the field of wristbands. More particularly, embodiments disclosed herein relate to modular wristband and sensor systems.

### BACKGROUND OF THE INVENTION

Current wristwatches/bands that contain electronic components within the bands require a permanent band selection, limiting the options for fashion and function. If bands are modular, then Bluetooth connectivity is required to communicate between top and bottom bands, risking connectivity issues. Creating bands that are able to be swapped and still conduct electricity introduces, inter alia, fashion variety. It also allows for bands of different length to be used so wristbands are not the only option. Sensors can have longer bands associated therewith for securing to larger areas of the body such as the legs, torso, biceps, head, neck, etc.

In consideration of wristbands that include sensors, the sensors are limited to the type of sensors that are initially included or embedded in the wristband upon obtaining the wristband. In other words, the sensor options for a typical wristband are not interchangeable, thereby limiting sensor options for the wristband.

Other limitations for conventional wristbands that include sensors exist such as limited space availability within the wristband in order to accommodate sensors and accompanying electronics, battery, wiring, etc.

Thus, it is desirable to provide a modular wristband and sensor system that is able to overcome the above disadvantages.

Advantages of the present invention will become more fully apparent from the detailed description of the invention hereinbelow.

### SUMMARY OF THE INVENTION

Embodiments are directed to a modular wristband and sensor system including: a sensor including a sensor conductive port; and a wristband including a wristband conductive port. The sensor conductive port is electrically and removably connected to the wristband conductive port.

Embodiments are also directed to a modular wristband and sensor system including: a wristband including a first sensor module connection port and a second sensor module connection port; a first sensor module configured to be removably connected to the first sensor module connection port; and a second sensor module configured to be removably connected to the second sensor module connection port. The first sensor module is communicatively connected to the second sensor module when the first sensor module and the second sensor module are respectively connected to the first sensor module connection port and the second sensor module connection port.

Embodiments are further directed to a method for using a modular wristband and sensor system. The method includes providing a modular wristband and sensor system including: a wristband including a first sensor module connection port and a second sensor module connection port; a first sensor module; and a second sensor module. The method also includes removably connecting the first sensor module to the first sensor module connection port; and removably connecting the second sensor module to the second sensor module connection port. The first sensor module is communicatively connected to the second sensor module when the first sensor module and the second sensor module are respectively connected to the first sensor module connection port and the second sensor module connection port.

Additional embodiments and additional features of embodiments for the modular wristband and sensor system are described below and are hereby incorporated into this section.

### BRIEF DESCRIPTION OF THE DRAWINGS

The foregoing summary, as well as the following detailed description, will be better understood when read in conjunction with the appended drawings. For the purpose of illustration only, there is shown in the drawings certain embodiments. It is understood, however, that the inventive concepts disclosed herein are not limited to the precise arrangements and instrumentalities shown in the figures. The detailed description will refer to the following drawings in which like numerals, where present, refer to like items.

FIG. 1 is a diagram of an architecture of an artificial intelligence-enabled health ecosystem;

FIG. 2A is a drawing illustrating an overall design of a four-piece modular two-sensor wristband with an inductive charging battery from a battery band;

FIG. 2B is a drawing illustrating a top/first/larger (swappable/modular) sensor module with conductive ports for charging and data transfer;

FIG. 2C is a drawing illustrating a modular flexible wristband with conductive ports;

FIG. 2D is a drawing illustrating an inductive charging battery band;

FIG. 2E is a drawing illustrating a wearable device (e.g., modular wristband and sensor system) including top/first/larger and bottom/second/smaller (swappable/modular) sensor modules connected to each other using a flexible/elastic fabric with embedded wiring to allow for data and/or power exchange;

FIG. 3A is a drawing illustrating a front perspective view of a modular wristband and sensor system including first and second (swappable/modular) sensor modules connected to the wristband;

FIG. 3B is a drawing illustrating a rear perspective view of the modular wristband and sensor system of FIG. 3A;

FIG. 3C is a drawing illustrating front and rear perspective views of the first and second sensor modules shown in FIG. 3A, connected to each other wirelessly;

FIG. 3D is a drawing illustrating a rear perspective view of the modular wristband and sensor system of FIG. 3C;

FIG. 3E is a drawing illustrating front and rear perspective views of the first and second sensor modules shown in FIG. 3C, connected to each other via wires (e.g., flex circuitry);

FIG. 3F is a drawing illustrating a rear perspective view of the modular wristband and sensor system of FIG. 3E;

FIG. 3G is a drawing illustrating front and rear perspective views of first and second (swappable/modular) sensor boards for the first and second sensor modules, respectively, shown in FIG. 3C, connected to each other via wires (e.g., flex circuitry);

FIG. 3H is a drawing illustrating a rear perspective view of the modular wristband and sensor system of FIG. 3G;

FIG. 3I is a drawing illustrating front and rear perspective views of first and second sensor module connection ports for respectively removably connecting the first and second sensor modules shown in FIG. 3C and the first and second sensor boards shown in FIG. 3G to the wristband shown in FIG. 3A;

FIG. 3J is a drawing illustrating a rear perspective view of the modular wristband and sensor system of FIG. 3I;

FIG. 3K is a drawing illustrating front and rear perspective views of a transparent view of the modular wristband and sensor system shown in FIG. 3A;

FIG. 3L is a drawing illustrating a rear perspective view of the modular wristband and sensor system of FIG. 3K;

FIG. 3M is a block diagram of the modular wristband and sensor system 300 of FIG. 3A.

### DETAILED DESCRIPTION OF THE INVENTION

It is to be understood that the figures and descriptions of the present invention may have been simplified to illustrate elements that are relevant for a clear understanding of the present invention, while eliminating, for purposes of clarity, other elements found in a typical modular wristband and sensor system or typical method of using a modular wristband and sensor system. Those of ordinary skill in the art will recognize that other elements may be desirable and/or required in order to implement the present invention. However, because such elements are well known in the art, and because they do not facilitate a better understanding of the present invention, a discussion of such elements is not provided herein. It is also to be understood that the drawings included herewith only provide diagrammatic representations of the presently preferred structures of the present invention and that structures falling within the scope of the present invention may include structures different than those shown in the drawings. Reference will now be made to the drawings wherein like structures are provided with like reference designations.

Before explaining at least one embodiment in detail, it should be understood that the inventive concepts set forth herein are not limited in their application to the construction details or component arrangements set forth in the following description or illustrated in the drawings. It should also be understood that the phraseology and terminology employed herein are merely for descriptive purposes and should not be considered limiting.

It should further be understood that any one of the described features may be used separately or in combination with other features. Other invented devices, systems, methods, features, and advantages will be or become apparent to one with skill in the art upon examining the drawings and the detailed description herein. It is intended that all such additional devices, systems, methods, features, and advantages be protected by the accompanying claims.

For purposes of this disclosure, the term “Bluetooth” may include and refer to BLE (Bluetooth Low energy) or other versions of Bluetooth such as Bluetooth 4, Bluetooth 5, etc., and thus, may all be used interchangeably.

A modular wristband and sensor system and method of using the same are disclosed. The system uses top and bottom sensor modules that may contain conductive ports for connection to wristbands' conductive ports. The wristbands' conductive ports are optionally electrically connected to wires embedded within each wristband segment. Those wires allow for the transfer of data and power between the bands and the top and bottom sensor modules. Having power and data conducted through wristbands into sensors make it possible for wristband sensors to have swappable bands while maintaining connectivity. There are currently no multi-sensor wristbands with conductive charging and data transfer capabilities on the market.

This device includes two wearable sensors with conductive ports, two band segments containing embedded wiring and conductive ports, and a sensor battery within at least one of the sensors. The sensor battery may be inductively charged via a battery from an adjacent battery band as described below, or may alternatively be directly conductively charged via a wire/USB port or via another known charging mechanism. As another alternative, inductive charging of the sensor battery may be employed via a charging pad similar to one used for mobile phones or smart watches. Once charged, the battery band may be secured next to the charging ports on the top wristband sensor, as that allows for inductive charging of the top sensor battery from the battery band battery. The top sensor along with its sensor battery connects to two wristband straps/segments via conductive ports which allow the transfer of both data and power. There may also be a bottom smaller sensor which contains a top and bottom set of conductive ports. The bottom sensor is powered by the top sensor battery via the wired connection through the wristbands. Data can be sent from the bottom sensor to the top sensor and vice versa via the wired wristbands as well.

This product is superior because it connects the two sensors via a wired connection embedded within the wristband segments, thereby removing the need for Bluetooth to connect the sensors to each other or an external device before the data can be used or combined. Additionally, this unique configuration allows the top sensor to contain a battery pack while the bottom sensor does not, meaning the system can reduce the overall footprint of the device. Finally, the sensor battery is charged via an inductive connection meaning there is no need for a physical insertion port for connecting to a wired charger, resulting in a reduced overall footprint of the device.

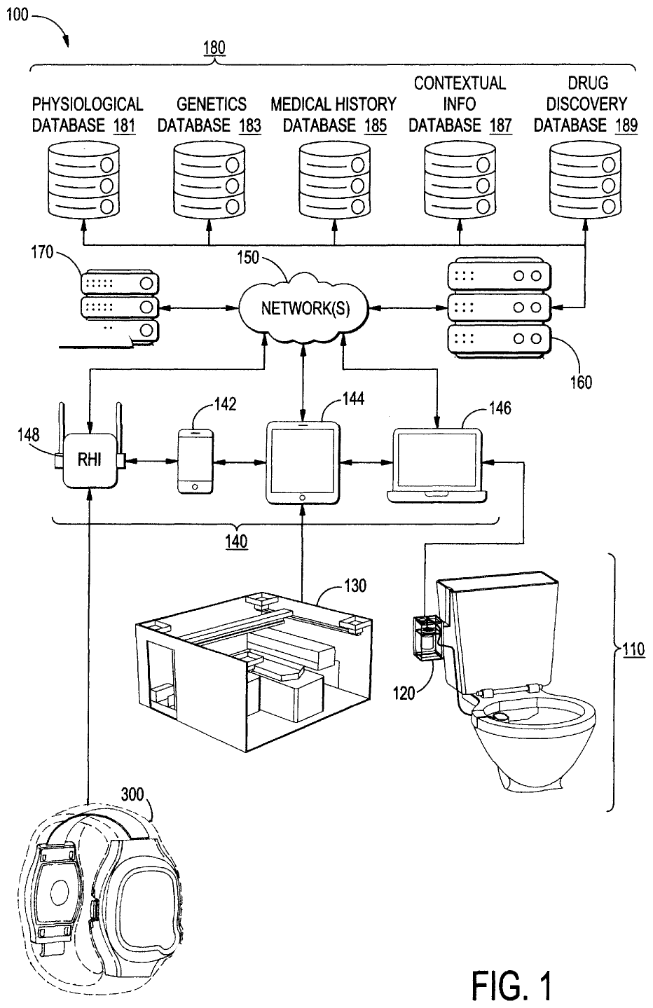

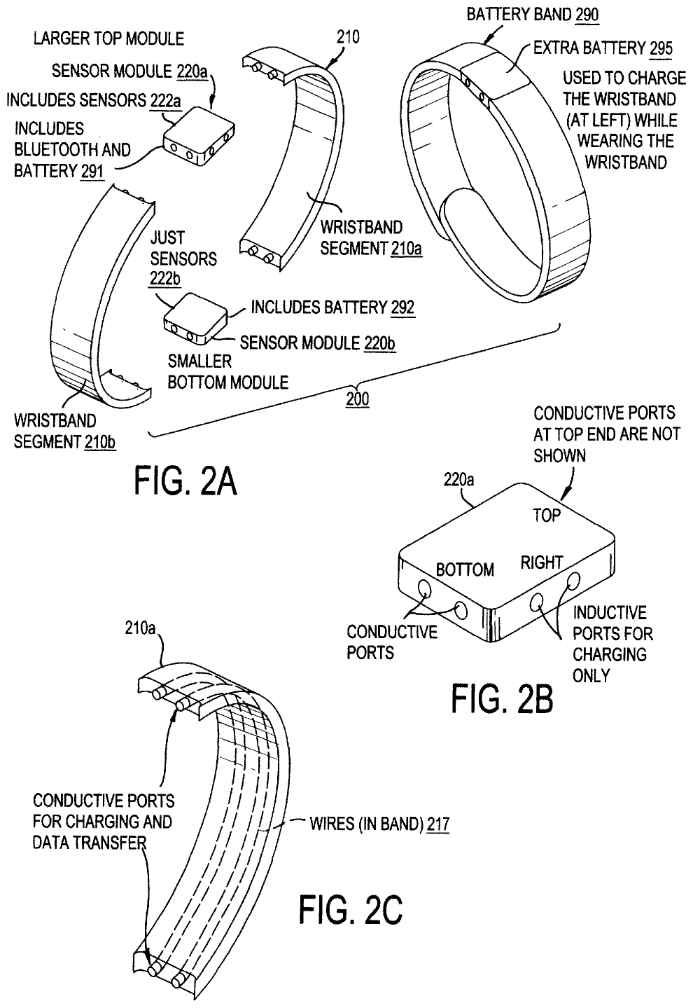

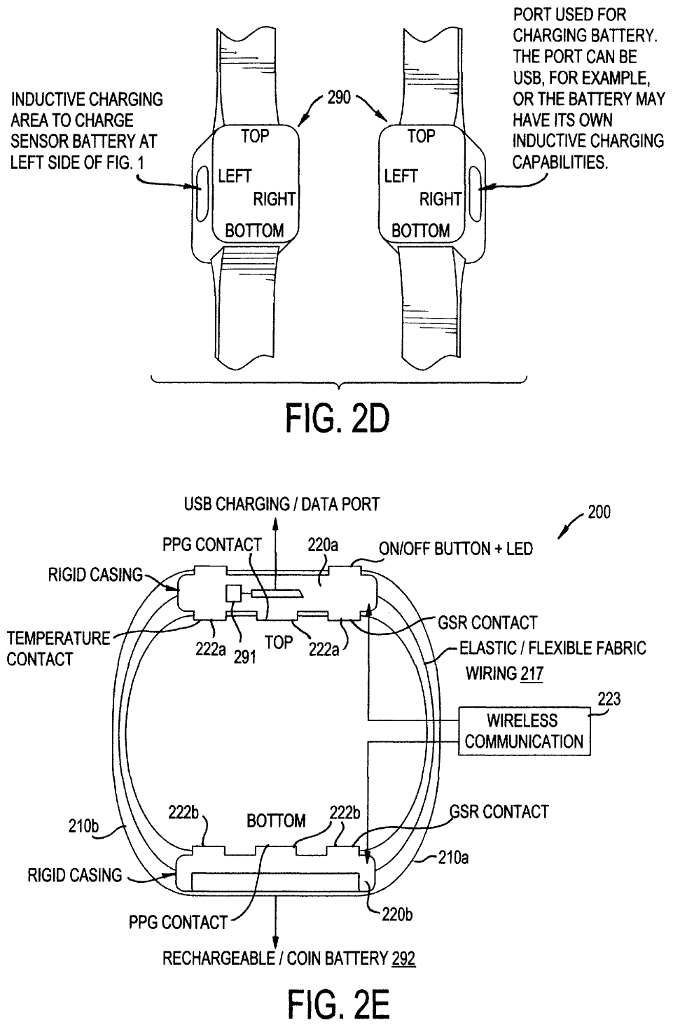

FIG. 1 is a diagram of an artificial intelligence-enabled health ecosystem 100 according to an exemplary embodiment.

As shown in FIG. 1, the AI-enabled health ecosystem 100 includes data acquisition devices 110 that communicate with a server 160 via local computing devices 140 and one or more computer networks 150. The server 160 stores data in non-transitory computer readable storage media 180 and may also receive data from third-party computer systems 170 (e.g., electronic health records systems) via the computer network(s) 150. In the embodiment of FIG. 1, the computer readable storage media 180 includes a physiological database 181, a genetics database 183, a medical history database 185, a contextual information database 187, and a drug discovery database 189.

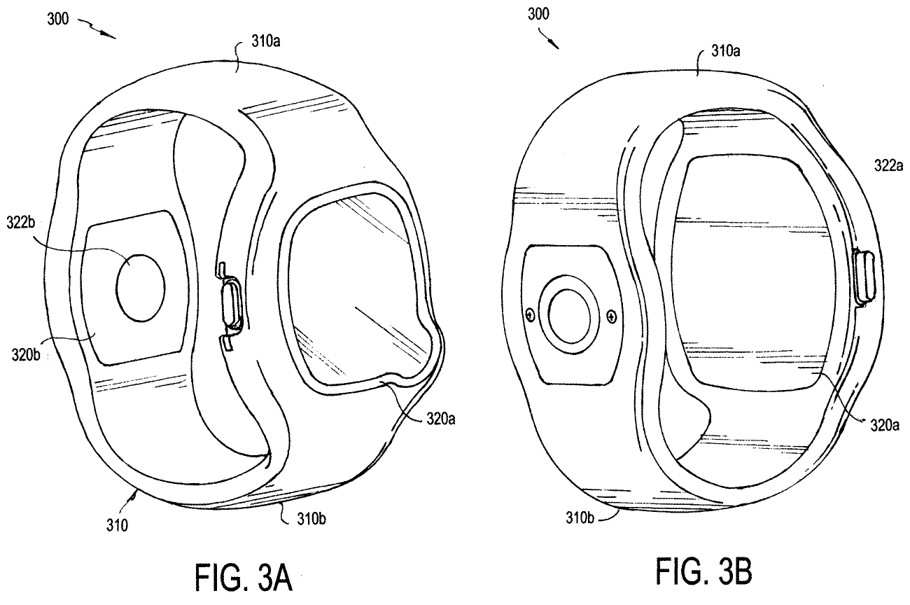

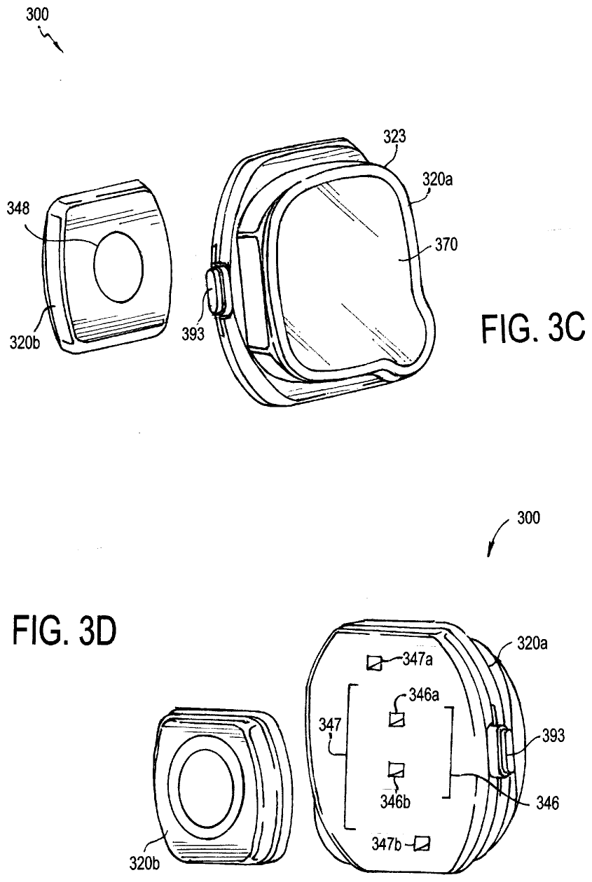

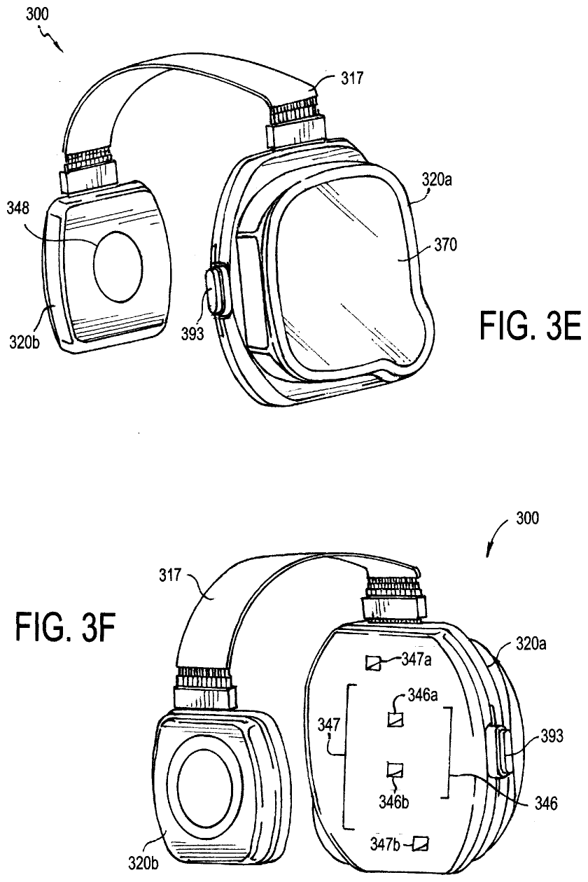

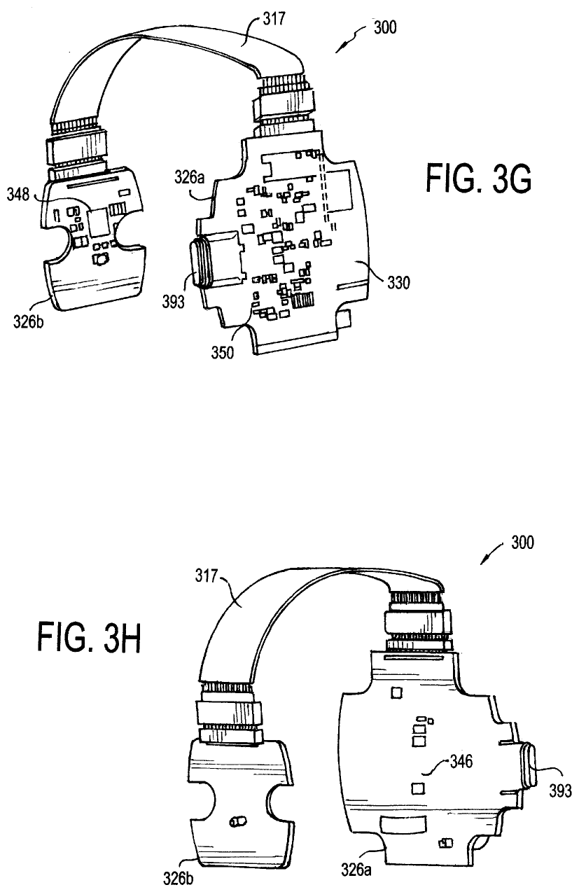

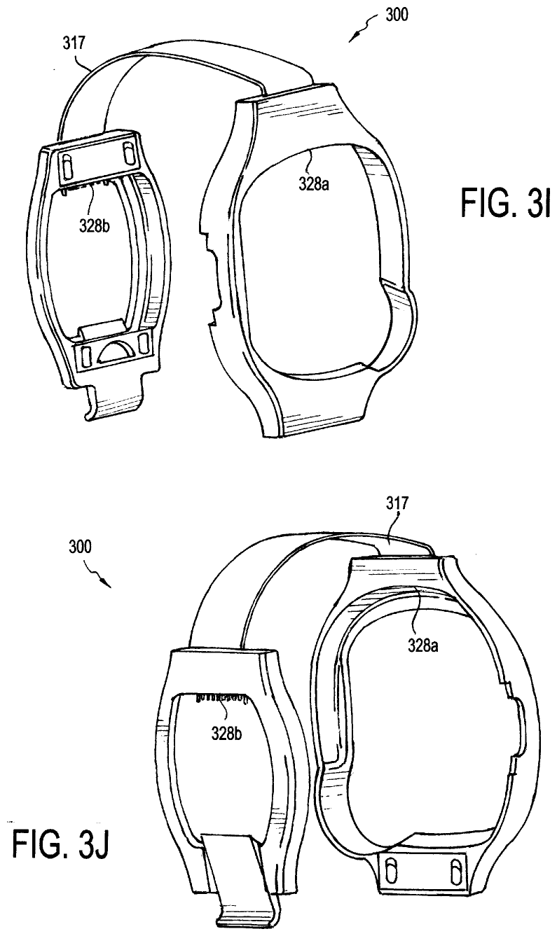

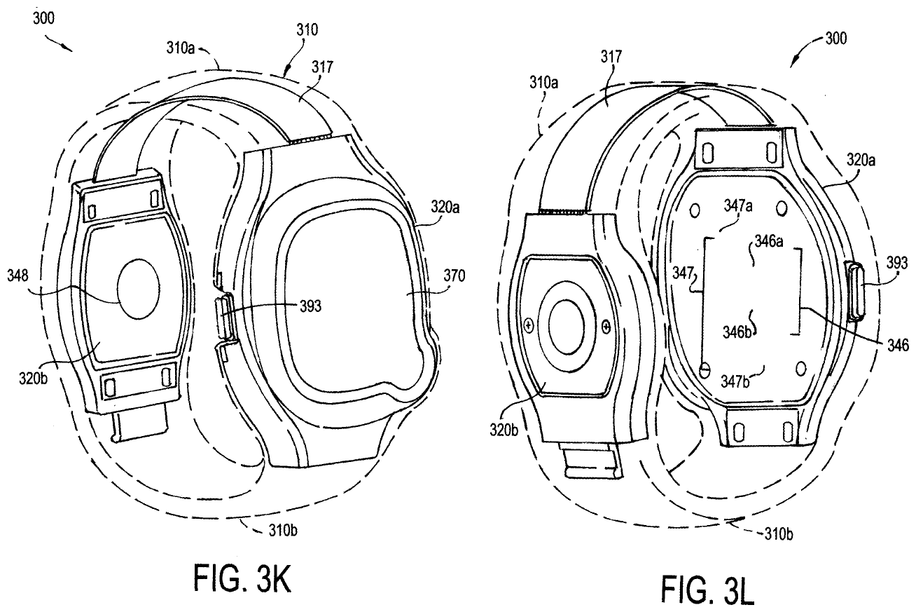

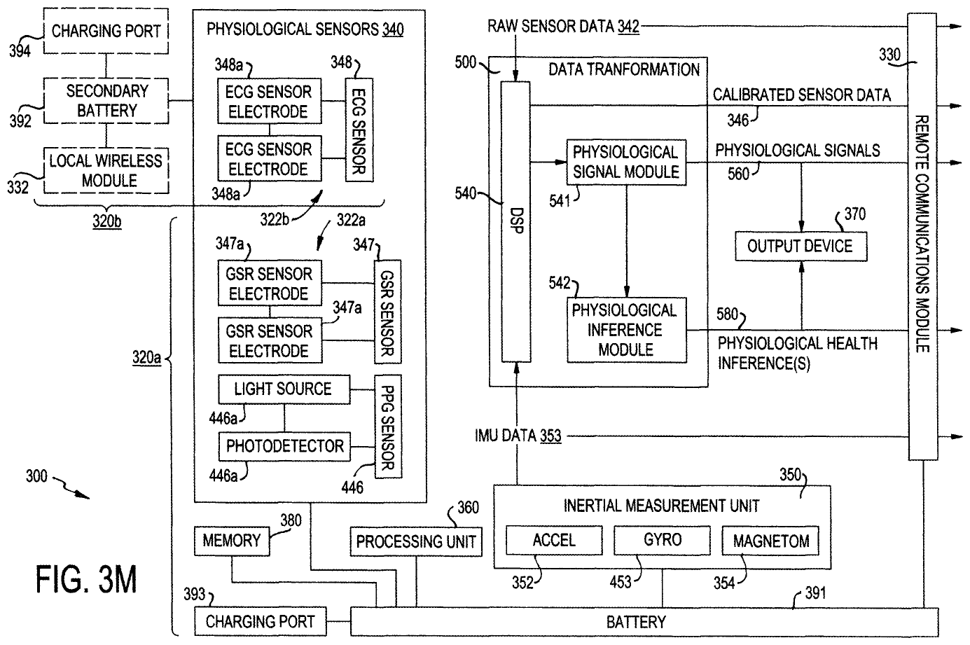

The data acquisition devices 110 may include a wearable health monitoring device such as modular wristband and sensor system 300, for example as described in detail below with reference to FIGS. 3A-3L, a biofluid analyzer (such as biofluid monitoring system 120, for example as described in co-pending U.S. patent application Ser. No. 17/833,842, filed Jun. 6, 2022), a genetic sequencer 130, etc. Another example of a wearable health monitoring device is a modular wristband and sensor system 200 as described in detail below with reference to FIGS. 2A-2E. As described below, each data acquisition device 110 may include multiple sensors.

The biofluid analyzer (e.g., biofluid monitoring system 120) may be any device capable of analyzing biofluid to identify biological markers of changing health and disease states. For example, the biofluid monitoring system 120 may capture biofluid and dispense the captured biofluid (e.g., a predetermined amount of biofluid) into a chemically coated disposable cartridge. The biofluid may and the chemical coating may initiate chemical reactions that cause color changes in the disposable cartridge that are indicative of biological markers. The biofluid monitoring system 120 may then measure those color changes (e.g., using a spectrometer) and output data indicative of those biological markers to a local computing device 140.

The genetic sequencer 130 may be any device capable of revealing the presence and quantity of ribonucleic acid (RNA). For example, the genetic sequencer 130 may collect a genetic sample (e.g., blood, urine, saliva, etc.), isolate RNA, create complementary deoxyribonucleic acid (cDNA), and sequence the RNA.

In preferred embodiments, the data acquisition devices 110 wirelessly communicate with the local computing devices 140 directly (e.g., using Bluetooth, Zigbee, etc.) or via a local area network (e.g., a Wi-Fi network). In other embodiments, a data acquisition device 110 may transfer data using a wired connection (e.g., a USB cable) or by storing data in a removable storage device (e.g., a USB flash memory device, a microSD card, etc.) that can be removed and inserted into a local computing device 140.

The local computing devices 140 may include any hardware computing device having one or more hardware computer processors that perform the functions described herein. For example, the local computing devices 140 may include smartphones 142, tablet computers 144, personal computers 146 (desktop computers, notebook computers, etc.), etc. The local computing devices 140 may also include dedicated processing devices 148 (installed, for example, in hospitals or other clinical settings) that form local access points to wirelessly receive data from wearable health monitoring devices (such as modular wristband and sensor systems 200, 300) and/or other data acquisition devices 110.

As described in detail below, the local computing devices 140 receive and process data from the data acquisition devices 110 and output the processed data to the server 160 via the one or more networks 150 (e.g., local area networks, cellular networks, the Internet, etc.). In some embodiments, the local computing devices 140 wirelessly communicate with each other, either via a local area network or using direct, wireless communication (e.g., via Bluetooth, Zigbee, etc.) to form a mesh network. Accordingly, in some embodiments, a data acquisition device 110 may output data to a child data acquisition device 110, which forwards that data to a parent data acquisition device 110 that forwards the data to the server 160. The server 160 may be any hardware computing device having one or more hardware computer processors that perform the functions described herein.

FIG. 2A is a drawing illustrating an overall design of a four-piece modular two-sensor wristband 210 (including wristband segments 210a, 210b) with an inductive charging battery from a battery band 290. More specifically, FIG. 2A is a drawing illustrating a four-piece modular wristband and sensor system 200 including two modular sensor modules 220a, 220b and two bands (i.e., separable/re-attachable wristband segments 210a, 210b), all of which have conductive charging capabilities. A battery may be positioned within the housing of either or both sensor modules 220a, 220b. Battery 291 may be positioned within the housing of larger sensor module 220a. Secondary battery 292 (if employed) may be positioned within the housing of smaller sensor module 220b. The battery 291 may itself be charged via conductive or inductive charging. The sizes and shapes of the sensor modules 220a, 220b may vary. In some instances, sensor module 220a may be smaller than sensor module 220b.

The larger sensor module 220a may include sensors 222a of any type such as for: heart rate, temperature, blood oxygen, blood flow (PPG), blood pressure, and galvanic skin response, and may contain a main battery 291 and Bluetooth connectivity. The smaller sensor module 220b may preferably only contain sensors 222b such as ECG (i.e., no battery or Bluetooth connectivity). The battery 295 from the battery band 290 is an additional/auxiliary battery used to charge (i.e., via inductive charging) the wristband while wearing the wristband.

FIG. 2B is a drawing illustrating a top/first (swappable/modular) sensor module 220a with conductive ports for charging and data transfer. More specifically, FIG. 2B is a drawing illustrating a top sensor module with conductive ports for charging and data transfer. The top sensor module is built with conductive ports on top and bottom of the sensor for connection to wiring in wristband segments/modules, allowing for power and data to run through the bands. The right side or left side of the top sensor may employ conductive or inductive charging ports for charging purposes.

FIG. 2C is a drawing illustrating a modular flexible wristband with conductive ports. More specifically, FIG. 2C is a drawing illustrating a modular flexible wristband segment with conductive ports for charging and data transfer. The figure shows one modular wristband segment 210a composed of flexible material with wiring 217 running through, and includes a pair of conductive ports on both ends of the wristband segment that are intended for connection to the corresponding sensors' conductive ports. The wristband segment's conductive ports are electrically connected to the wiring running through the wristband segment.

FIG. 2D is a drawing illustrating an inductive charging battery band 290. More specifically, FIG. 2D is a drawing illustrating a wire and inductive charging battery band. The battery band includes a wired charging point on the left side and inductive charging connection(s) on the right side. These charging connections may alternatively be reversed, may both be wired, or may both be inductive. The battery band is placed on a user's wrist alongside and adjacent to the modular wristband and sensors. The battery from the battery band may provide power to either sensor via the wired or inductive charging ports, when the battery is adjacent to (or close to, in the case of inductive charging) the corresponding sensor to be powered.

In another embodiment, two batteries may be provided within the battery band, i.e., one battery for each sensor.

As another alternative, the modular wristband and sensor system may include only a single sensor. In that embodiment, the battery band provides the power directly to the single sensor, and no wires running through the sensor's band would be necessary.

The top and bottom sensors may be interchangeable with each other or with other sensors of different types and sizes. Either or both the top and bottom sensors may be comprised of any of the sensor types described below. The flexible wristband segments (having the wires embedded therein) may be comprised of liquid silicone rubber or another flexible material suitable for contact with a user's skin. Other wireless standards other than Bluetooth may be employed, such as Zigbee or Wi-Fi.

The wearable wristband device includes a core sensor pack/module (note the large and small sensors mentioned throughout this disclosure may include a “sensor pack”) on the top of the wristband that contains 5 main sensors for: heart rate, temperature, blood oxygen, blood pressure, and galvanic skin response. The wearable wristband device also contains a sensor module pack on the bottom of the wristband which can be customized and swapped out for specific sensor desires. The wearable wristband device also contains Bluetooth connectivity between the top and bottom sensor pack to communicate between sensor modules. For instance, a sensor pack customized to monitor electrocardiogram readings, GPS, biokinetics, body composition, bioelectric impedance analysis, electromyograms, electroencephalograms, chemotherapy levels, glucose levels, ketone levels, organic compounds in exhaled breath, blood alcohol levels, biomarkers, genetic content, and/or hydration levels could be swapped into the area of the wristband that is in contact with the bottom of the wrist. The wearable wristband device collects data from its sensors and sends the data (e.g., via Bluetooth) to a local computing device 140 to be analyzed via a software application (e.g., a smartphone application) that utilizes a model developed via a machine learning pipeline. Early use of the wearable device and accompanying application will indicate either an individual's health or their divergence from health. Over time, the application will evolve to indicate more specific indications as the machine learning algorithms become more customized to the individual's health baseline. Data will only be passed to the patient's health care provider with the patient's permission.

What it is: A wearable device with multiple sensor packs, one as the core sensor pack, and at least one with modular capabilities.

What it does: It measures physiological data from a PPG sensor, as well as a temperature, and galvanic skin response sensor, as well as interchangeable customizable sensors on the bottom sensor pack, and transmits that physiological data to a local computing device 140.

Why it is needed: It is needed to provide a low cost alternative to the current medical-grade wearable devices on the market, as well as to provide customization to the sensor packs in use, so that sensor packs could be designed for use cases and added to the bottom, modular, sensor pack location.

This invention solves the problem of wanting to customize the sensor pack or group of sensors for individual applications.

The wearable device may be a custom medical-grade wearable device which has a unique wristband-like design with the sensors and the battery packed into rigid casings within the top and bottom modules; and the wiring is embedded into the elastic fabric for communications and connectivity.
Top module:
   Sensors that are absolutely required and are here to stay on a more permanent basis could be placed in the top module
   Contains communications infrastructure to support streaming sensor data to a mobile device via Bluetooth
   Contains a power management system (including the battery)
   Not intended to change across clients
Bottom module:
   Extensible module customizable per client
   Could contain additional sensors which would use the communications infrastructure in the top module to stream data
   Could be used as a communications hub to connect and stream data from external sensors using the communications infrastructure in the top module
   Could contain additional battery to augment the battery life of the wearable
Fabric:
   Flexible elastic material to enable snug fit and idiot-proof the sensor placement on the wrist
   Conductive material to allow power and communications between the top and bottom modules

The wearable device may be a custom medical-grade wearable device. It incorporates a collection of physiological sensors including ECG, PPG, Galvanic skin response (GSR), accelerometer (e.g., 3-axis type), gyroscope (e.g., 3-axis type), magnetometer (e.g., 3-axis type), and skin temperature. It has a flexible and stretchable wristband-like design to enable a snug fit and reliable sensor contact. This band also includes embedded wiring for connectivity to the bottom module for data and power exchange. It has a smart power management system to maximize the battery life of the device. The wearable device will have the ability to identify and smartly handle durations of intermittent connectivity by leveraging on-device flash-storage. It has a communications module that supports connectivity protocols including, for example, Bluetooth Low Energy, Zigbee, Wi-Fi, and/or Thread to transfer data wirelessly over to a local computing device 140.

The local computing device 140 will in turn act as the gateway into the server 160 of the AI-enable health ecosystem 100, where machine learning models for a variety of applications are trained. Those models are then deployed back to the local computing device 140 or modular wristband and sensor system 200 to provide personalized health insights about the individual wearing the modular wristband and sensor system 200 to make on-device actionable insights in real-time including finding anomalies and early-detection of disease markers. Those insights are made available at the wearable device user's fingertips via a companion application that allows individuals to make decisions quicker. This companion application (running on the local computing device 140) will streamline data acquisition in fast-paced settings (including emergency rooms, manufacturing facilities etc.), allowing for more accurate (with our proprietary DSP algorithms), secure (HIPAA compliant data transport) and robust data acquisition (ensuring no data loss/corruption) enabling significantly reduced time between acquisition and analysis (for machine learning (ML)).

What it does: Reliable data acquisition

Why it is needed: In addition to being a platform for reliable data acquisition, the wearable device (and the app) is a part of an AI-enabled health ecosystem 100, which is an integrated solution for:
Researchers and clinicians to conduct studies that could leverage AI for digital therapeutics and early detection of disease.
Doctors and care providers to remotely monitor their patients over an extended period of time.
Factory operators (including in the mining and manufacturing industries) to track the health and wellbeing of their workers while performing physically demanding tasks.
Individual consumers to monitor and track their own health insights over time to make healthy life choices.

What problems are solved:
No commercially available medical-grade wearable device enables the collection of raw data from optical and other physiological sensors. We provide this ability, enabling us to develop better ML and AI algorithms to tackle problems in the field of digital therapeutics.
Other medical grade wearables with similar functionality are an order of magnitude more expensive. We make this possible by pushing a lot of the complexity to the app where a majority of the heavy lifting happens (e.g. DSP).

Example of how the system is superior and/or different:
1. Unique design (top module+bottom module+flexible wristband).
2. Flexible wristband-like design to allow for a perfect fit with consistent skin contact and adequate thermal coupling to enable reliable data acquisition.
3. Extensible bottom module customizable per client.
   a. Could contain additional sensors which would use the communications infrastructure in the top module to stream data.
   b. Could be used as a communications hub to connect and stream data from additional external sensors using the communications infrastructure in the top module.
   c. Could contain additional battery to augment the battery life of the wearable.
4. Top module streams a unique collection of physiological data
   a. Raw PPG data (IR, Red and Green)—as far as I know, no commercially available device streams raw PPG data
   b. GSR
   c. IMU (accelerometer, gyroscope, magnetometer)
   d. Skin temperature

FIG. 2E is a drawing illustrating a wearable device (e.g., modular wristband and sensor system 200) including top/first and bottom/second (swappable/modular) sensor modules 220a, 220b connected to each other using a flexible/elastic fabric band with embedded wiring 217 within wristband segments 210a, 210b to allow for data and/or power exchange. More specifically, FIG. 2E is a drawing illustrating a wearable device including top and bottom sensor modules connected to each other using a flexible/elastic fabric with embedded wiring to allow for data and power exchange. Note that the battery may be in either or both of the top sensor module 220a or the bottom sensor module 220b.

With reference to FIGS. 2A-2E, embodiments are directed to a modular wristband and sensor system including: a sensor including a sensor conductive port; and a wristband including a wristband conductive port. The sensor conductive port is electrically and removably connected to the wristband conductive port.

In an embodiment, the sensor is a first sensor and the wristband is a first wristband segment. The system further includes a second sensor and a second wristband segment. Each wristband segment conductively connects to both sensors and may do so on opposing sides of the sensors. The first sensor is conductively connected to the second sensor via wires embedded within the first wristband segment and, optionally, the second wristband segment. The wires allow for power and data transfer to be provided to the sensors. In an alternative embodiment, the sensor modules 220a, 220b wirelessly communicate with each other via wireless communication 223 (see, for example, FIG. 2E) which includes Bluetooth, Zigbee, or Wi-Fi.

In an embodiment, the system further includes a battery band including a battery. The battery band is configured to be placed on a user's wrist alongside and adjacent to the wristband such that the battery is adjacent to the sensor to provide power to the sensor.

FIGS. 3A-3B are drawings respectively illustrating front and rear perspective views of a modular wristband and sensor system 300 including first and second (swappable/modular) sensor modules 320a, 320b connected to the wristband 310. The wristband 310 includes wristband segments 310a, 310b.

FIGS. 3C-3D are drawings respectively illustrating front and rear perspective views of the first and second sensor modules 320a, 320b shown in FIG. 3A, connected to each other wirelessly.

FIGS. 3E-3F are drawings respectively illustrating front and rear perspective views of the first and second sensor modules 320a, 320b shown in FIG. 3C, connected to each other via wires 317 (e.g., flex circuitry).

FIGS. 3G-3H are drawings respectively illustrating front and rear perspective views of first and second (swappable/modular) sensor boards 326a, 326b for the first and second sensor modules 320a, 320b, respectively, shown in FIG. 3C, connected to each other via wires 317 (e.g., flex circuitry).

FIGS. 3I-3J are drawings respectively illustrating front and rear perspective views of first and second sensor module connection ports 328a, 328b for respectively removably connecting the first and second sensor modules 320a, 320b shown in FIG. 3C and the first and second sensor boards 326a, 326b shown in FIG. 3G to the wristband shown in FIG. 3A.

FIGS. 3K-3L are drawings respectively illustrating front and rear perspective transparent views of the modular wristband and sensor system 300 shown in FIG. 3A. As shown in FIGS. 3K-3L, the wearable health monitoring device 300 includes two sensor modules 320a and 320b connected to wristband segments 310a and 310b to form a wristband 310. The sensor module 320a includes an output device 370 (see FIG. 3M, in this embodiment, a display).

In the embodiment of FIGS. 3K-3L, the sensor module 320a includes a PPG sensor 346 (having a light source 346a and a photodetector 346b) and a GSR sensor 347 (having GSR sensor electrodes 347a and 347b) and the sensor module 320b includes an ECG sensor 348 (having ECG sensor electrodes 348a and 348b shown in FIG. 3M and described below). However, in other embodiments, the wearable health monitoring device 300 may include any of a number of different physiological and other sensors. In fact, as described below, either or both of the sensor modules 320a and 320b may be removable and replaceable, enabling the wearable health monitoring device 300 to include different sensors as needed for specific applications. For example, for an individual or organization in the mining industry, the wearable health monitoring device 300 may include a sensor module that includes a number of gas sensors.

In the embodiment of FIGS. 3K-3L, the sensor module 320a includes a charging port 393 for charging a battery 391 (shown in FIG. 3M and described below) that provides power to the sensor module 320a and the sensor module 320b via wiring 317 (e.g., flex circuitry) in the wristband 310. However, other embodiments may not include wiring 317. Instead, in those embodiments, the sensor module 320b may wirelessly communicates with the sensor module 320a via a direct, short range communication protocol (e.g., Zigbee, Bluetooth, etc.) and may include a battery and a charging port for providing power to the battery (as described below with reference to FIG. 3M).

FIGS. 3C-3D are views of the sensor modules 320a and 320b (removed from the wristband segments 310a and 310b) according to an exemplary embodiment. FIGS. 3E-3F are views of the sensor modules 320a and 320b and wiring 317 (removed from the wristband segments 310a and 310b) according to an exemplary embodiment. FIGS. 3G-3H are views of a sensor board 326a of the sensor module 320a and a sensor board 326b of the sensor module 320b according to an exemplary embodiment. In the embodiment of FIG. 3G, the sensor module 320a also includes an inertial measurement unit 350 and a communications module 330.

FIGS. 3I-3J are views of a sensor module connection port 328a for the sensor module 320a and a sensor module connection port 328b for the sensor module 320b. As shown in FIGS. 3I and 3J, the connection ports 328a, 328b enable sensor modules to be removed, reconnected, and/or replaced with a different sensor module having different physiological or other sensors.

With reference to FIGS. 3A-3L, embodiments are also directed to a modular wristband and sensor system 300 including: a wristband 310 including a first sensor module connection port 328a and a second sensor module connection port 328b; a first sensor module 320a configured to be removably connected to the first sensor module connection port 328a; and a second sensor module 320b configured to be removably connected to the second sensor module connection port 328b. The first sensor module 320a is communicatively connected to the second sensor module 320b when the first sensor module 320a and the second sensor module 320b are respectively connected to the first sensor module connection port 328a and the second sensor module connection port 328b.

In an embodiment, the first sensor module 320a is distant from the second sensor module 320b along the wristband 310.

In an embodiment, the first sensor module 320a houses a battery 391. A battery may alternatively be housed in the second sensor module 320b.

In an embodiment, the first sensor module 320a is connected to the second sensor module 320b via wires 317 embedded within the wristband 317, and wherein the wires 317 provide power from the battery 391 to the second sensor module 320b. The wires 317 may further provide communication between the first sensor module 320a and the second sensor module 320b.

In an embodiment, the first sensor module 320a includes multiple sensors 322a (see FIG. 3A, shown are two sensors which are preferably different types from each other) and the second sensor module 320b includes a second sensor 322b (see FIG. 3A) which is different than the sensors 322a.

In an embodiment, the first sensor and the second sensor each includes a sensor selected from the group consisting of electrocardiogram (ECG), Photoplethysmography (PPG), galvanic skin response (GSR), accelerometer, gyroscope, magnetometer, and skin temperature, and combinations thereof.

In an embodiment, the first sensor module 320a is configured to wirelessly communicate with the second sensor module 320b. The wireless communication 323 (see, for example, FIG. 3C) may include Bluetooth, Zigbee, or Wi-Fi.

With further reference to FIGS. 3A-3L, embodiments are further directed to a method for using a modular wristband and sensor system. The method includes providing a modular wristband and sensor system including: a wristband including a first sensor module connection port and a second sensor module connection port; a first sensor module; and a second sensor module. The method also includes removably connecting the first sensor module to the first sensor module connection port; and removably connecting the second sensor module to the second sensor module connection port. The first sensor module is communicatively connected to the second sensor module when the first sensor module and the second sensor module are respectively connected to the first sensor module connection port and the second sensor module connection port.

FIG. 3M is a block diagram of the modular wristband and sensor system 300 according to exemplary embodiments. The components set forth in FIG. 3M may also be applicable to modular wristband and sensor system 200 described above.

As shown in FIG. 3M, the modular wristband and sensor system 300 includes two sensor modules 320a and 320b, each with one or more sensors 322a and 322b. The sensors 322a and 322b include physiological sensors 340. The modular wristband and sensor system 300 also includes a remote communications module 330, an inertial measurement unit 350, a hardware computer processing unit 360, output device(s) 370, memory 380, a battery 391, a charging port 393, and data transformation modules 500.

In the embodiment of FIG. 3M, the remote communications module 330 enables the modular wristband and sensor system 300 to output data for transmittal to a local computing device 140. The remote communications module 330 may include, for example, a module for short range, direct, wireless communication (e.g., Bluetooth, Zigbee, etc.) and/or a module for communicating via a local area network (e.g., Wi-Fi). In other embodiments, the remote communications module 330 may enable the modular wristband and sensor system 300 to bidirectionally communicate with the server 160 (see FIG. 1) via the one or more networks 150.

The output device 370 may include a display (e.g., as shown in FIGS. 3A, 3C, 3E, and 3K), a speaker, a haptic feedback device, etc. The memory 380 may include any non-transitory computer readable storage media (e.g., a hard drive, flash memory, etc.). The processing unit 360 may include any hardware computing device suitably programmed to perform the functions described herein (e.g., a central processing unit executing instructions stored in the memory 380, a state machine, a field programmable array, etc.).

The battery 391 provides power to the sensor module 320a. In some embodiments, the battery 391 also provides power to the sensor module 320b via the wires 317 described above. In those embodiments, the sensor module 320b transfers data (e.g., output by the ECG sensor 348) to the sensor module 320a via the wires 317. In other embodiments, however, the sensor module 320b wirelessly communicates with the sensor module 320a via a direct, short range communication protocol (e.g., Zigbee). In those embodiments, the sensor module 320b may also include a local wireless module 332 for sending data to the sensor module 320a. When power is not transmitted through wires 317, a secondary battery 392 for providing power to the sensor module 320b, and a charging port 394 for providing power to the secondary battery 392 may be employed.

The charging port 393 (and the charging port 394) may each be a hardware port for receiving electrical power (e.g., a universal serial bus port, an inductive charging port, etc.).

The physiological sensors 340 may include any device capable of sensing data indicative of a physiological or biochemical condition of the wearer. In the embodiment of FIG. 3M, the physiological sensors 340 include a PPG sensor 446 having a light source 446a and a photodetector 446b, a GSR sensor 347 having GSR sensor electrodes 347a and 347b, and an ECG sensor 348 having ECG sensor electrodes 348a and 348b. The PPG sensor 446 may be any device capable of obtaining (e.g., optically) a plethysmogram that can be used to detect blood volume changes in the microvascular bed of tissue. The GSR sensor 347 may be any device capable of sensing the electrical conductance of the skin (i.e., the galvanic skin response). The ECG sensor 348 may be any device capable of sensing electrical signals generated by the beating heart of the wearer.

The inertial measurement unit 350 may be any device capable of measuring and reporting the specific force and angular rate of the modular wristband and sensor system 300. The inertial measurement unit 350 may also measure and report the orientation of the modular wristband and sensor system 300. In the embodiment of FIG. 3M, the inertial measurement unit 350 includes an accelerometer 352 (e.g., a 3-axis accelerometer), a gyroscope 453, and a magnetometer 354.

The inertial measurement unit 350 outputs IMU data 353 indicative of the movement of the modular wristband and sensor system 300. The physiological sensors 340 output raw sensor data 342 indicative of a physiological or biochemical condition of the user. The remote communications module 330 outputs the IMU data 353 and the raw sensor data 342 for transmittal to the server 160 (e.g., via a local computing device 140).

In some embodiments, the modular wristband and sensor system 300 also includes data transformation modules 500. In the embodiment of FIG. 3M, for example, the modular wristband and sensor system 300 includes a digital signal processing module 540 that performs digital signal processing (DSP) on the raw sensor data 342 (e.g., to remove motion artifacts and/or noise) and generates calibrated sensor data 346, a physiological signal module 541 that identifies physiological signals 560 based on the calibrated sensor data 346, and a physiological inference module 542 that makes physiological health inferences 580 based on those physiological signals 560. The remote communications module 330 outputs the calibrated sensor data 346, the physiological signals 560, and any physiological health inferences 580 for transmittal to the server 160 (e.g., via a local computing device 140). In some embodiments, the physiological signals 560 may also be output to the user via an output device 370 (e.g., displayed to the user via a display). Physiological health inferences 580 may also be output to the user via an output device 370. For example, a visual, audible, and/or tactile alert may output to the user via display, a speaker, and/or a haptic feedback device.

The method steps in any of the embodiments described herein are not restricted to being performed in any particular order. Also, structures or systems mentioned in any of the method embodiments may utilize structures or systems mentioned in any of the device/system embodiments. Such structures or systems may be described in detail with respect to the device/system embodiments only but are applicable to any of the method embodiments.

Features in any of the embodiments described in this disclosure may be employed in combination with features in other embodiments described herein, such combinations are considered to be within the spirit and scope of the present invention.

The contemplated modifications and variations specifically mentioned in this disclosure are considered to be within the spirit and scope of the present invention.

More generally, even though the present disclosure and exemplary embodiments are described above with reference to the examples according to the accompanying drawings, it is to be understood that they are not restricted thereto. Rather, it is apparent to those skilled in the art that the disclosed embodiments can be modified in many ways without departing from the scope of the disclosure herein. Moreover, the terms and descriptions used herein are set forth by way of illustration only and are not meant as limitations. Those skilled in the art will recognize that many variations are possible within the spirit and scope of the disclosure as defined in the following claims, and their equivalents, in which all terms are to be understood in their broadest possible sense unless otherwise indicated.

1. A modular wristband and sensor system comprising:
a sensor comprising a sensor conductive port; and
a wristband comprising a wristband conductive port;
wherein the sensor conductive port is electrically and removably connected to the wristband conductive port.

2. The system of claim 1, wherein the sensor is a first sensor and the wristband is a first wristband segment, wherein the system further comprises a second sensor and a second wristband segment, wherein each wristband segment conductively connects to both sensors, wherein the first sensor is conductively connected to the second sensor via wires embedded within the first wristband segment, and wherein the wires allow for power and data transfer to be provided to the sensors.

3. The system of claim 1, wherein the sensor is a first sensor and the wristband is a first wristband segment, wherein the system further comprises a second sensor and a second wristband segment, wherein each wristband segment conductively connects to both sensors on opposing sides of the sensors, wherein the first sensor is conductively connected to the second sensor via wires embedded within the first and second wristband segments, and wherein the wires allow for power and data transfer to be provided to the sensors.

4. The system of claim 1, wherein the system further comprises a battery band comprising a battery, wherein the battery band is configured to be placed on a user's wrist alongside and adjacent to the wristband such that the battery is adjacent to the sensor to provide power to the sensor.

5. A modular wristband and sensor system comprising:
a wristband comprising a first sensor module connection port and a second sensor module connection port;
a first sensor module configured to be removably connected to the first sensor module connection port; and
a second sensor module configured to be removably connected to the second sensor module connection port;
wherein the first sensor module is communicatively connected to the second sensor module when the first sensor module and the second sensor module are respectively connected to the first sensor module connection port and the second sensor module connection port.

6. The system of claim 5, wherein the first sensor module is distant from the second sensor module along the wristband.

7. The system of claim 5, wherein the first sensor module houses a battery.

8. The system of claim 7, wherein the first sensor module is connected to the second sensor module via wires embedded within the wristband, and wherein the wires provide power from the battery to the second sensor module.

9. The system of claim 8, wherein the wires further provide communication between the first sensor module and the second sensor module.

10. The system of claim 5, wherein the first sensor module comprises a first sensor and the second sensor module comprises a second sensor which is different than the first sensor.

11. The system of claim 10, wherein the first sensor and the second sensor each comprise a sensor selected from the group consisting of electrocardiogram (ECG), Photoplethysmography (PPG), galvanic skin response (GSR), accelerometer, gyroscope, magnetometer, and skin temperature, and combinations thereof.

12. The system of claim 5, wherein the first sensor module is configured to wirelessly communicate with the second sensor module.

13. The system of claim 12, wherein the wireless communication comprises Bluetooth, Zigbee, or Wi-Fi.

14. A method for using a modular wristband and sensor system, the method comprising:
providing a modular wristband and sensor system comprising:
a wristband comprising a first sensor module connection port and a second sensor module connection port;
a first sensor module; and
a second sensor module;
removably connecting the first sensor module to the first sensor module connection port; and
removably connecting the second sensor module to the second sensor module connection port;
wherein the first sensor module is communicatively connected to the second sensor module when the first sensor module and the second sensor module are respectively connected to the first sensor module connection port and the second sensor module connection port.

15. The method of claim 14, wherein the first sensor module is distant from the second sensor module along the wristband.

16. The method of claim 14, wherein the first sensor module houses a battery.

17. The method of claim 16, wherein the first sensor module is connected to the second sensor module via wires embedded within the wristband, and wherein the wires provide power from the battery to the second sensor module.

18. The method of claim 17, wherein the wires further provide communication between the first sensor module and the second sensor module.

19. The method of claim 14, wherein the first sensor module comprises a first sensor and the second sensor module comprises a second sensor which is different than the first sensor.

20. The method of claim 19, wherein the first sensor and the second sensor each comprise a sensor selected from the group consisting of electrocardiogram (ECG), Photoplethysmography (PPG), galvanic skin response (GSR), accelerometer, gyroscope, magnetometer, and skin temperature, and combinations thereof.

21. The method of claim 14, wherein the first sensor module wirelessly communicates with the second sensor module.

22. The method of claim 21, wherein the wireless communication comprises Bluetooth, Zigbee, or Wi-Fi.
